(19) United States
(12) Patent Application Publication
(10) Pub. No.: US 2018/0153155 A1
(43) Pub. Date: Jun. 7, 2018

(54) METHOD FOR CRYOPRESERVATION OF CARDIOCYTES DERIVED FROM PLURIPOTENT STEM CELLS OR MESENCHYMAL STEM CELLS DERIVED FROM ADIPOSE TISSUE OR BONE MARROW

(71) Applicants: TERUMO KABUSHIKI KAISHA, Tokyo (JP); OSAKA UNIVERSITY, Osaka (JP)

(72) Inventors: Fumiya OHASHI, Ashigarakami-gun (JP); Shigeru MIYAGAWA, Osaka (JP); Yoshiki SAWA, Osaka (JP); Shigeo MASUDA, Osaka (JP); Satsuki FUKUSHIMA, Osaka (JP); Atsuhiro SAITO, Osaka (JP)

(73) Assignees: TERUMO KABUSHIKI KAISHA, Tokyo (JP); OSAKA UNIVERSITY, Osaka (JP)

(21) Appl. No.: 15/872,157

(22) Filed: Jan. 16, 2018

Related U.S. Application Data
(63) Continuation of application No. PCT/JP2016/070816, filed on Jul. 14, 2016.

(30) Foreign Application Priority Data
Jul. 15, 2015 (JP) 2015-141181

(51) Int. Cl. A01N 1/02; G01N 33/50; C12N 5/077; A61K 35/545

(52) U.S. Cl. CPC A01N 1/0215; G01N 33/5073; C12N 5/0653; A01N 1/0221; A61K 35/545; C12N 5/0657

(57) ABSTRACT

A method is disclosed for cryopreservation of cardiocytes derived from pluripotent stem cells or mesenchymal stem cells derived from adipose tissue or bone marrow, the method maintaining the function of the cardiocytes derived from differentiated pluripotent stem cells or mesenchymal stem cells derived from adipose tissue or bone marrow, and yet reducing the possibility for tumorigenesis of undifferentiated pluripotent stem cells or mesenchymal stem cells derived from adipose tissue or bone marrow. A method is also disclosed for cryopreservation of cardiocytes derived from pluripotent stem cells or mesenchymal stem cells (derived from adipose tissue or bone marrow, the method including dissociating cells from a cell population which has been induced to differentiate into cardiocytes from pluripotent stem cells or mesenchymal stem cells derived from adipose tissue or bone marrow.

### CROSS-REFERENCES TO RELATED APPLICATIONS

This application is a continuation of International Application No. PCT/JP2016/070816 filed on Jul. 14, 2016, which claims priority to Japanese Application No. 2015-141181 filed on Jul. 15, 2015, the entire contents of which are incorporated herein by reference.

### TECHNICAL FIELD

The present disclosure relates to a method for cryopreservation of cardiocytes derived from pluripotent stem cells or mesenchymal stem cells derived from adipose tissue or bone marrow, a method for producing a sheet-shaped cell culture containing the cardiocytes, and an application of the sheet-shaped cell culture.

### BACKGROUND DISCUSSION

Despite the recent innovative advance in therapy for heart diseases, no medical treatment system has been established for severe cardiac incompetence. It is believed that patients suffering from severe cardiac incompetence will effectively recover cardiac function through cell transplantation. Clinical application and research for this purpose have started with cardiocytes derived from induced pluripotent stem cells (iPS cells) and autoskeletal muscle blast cells. Such efforts have resulted in a cell culture and a method for production thereof. This cell culture contains cells derived from any other parts of the body than cardiac muscle and takes on a three-dimensional shape applicable to the heart. Moreover, this cell culture is obtained by a temperature-responsive culture dish, which is based on the system engineering (JPT2007528755). Unfortunately, the sheet-shaped cell culture prepared by using a temperature-responsive culture dish is fragile and easy to break, and hence it involves difficulties in transportation.

There is also a known method for producing a sheet-shaped cell culture from cells which have undergone freezing and thawing (PCT Patent Publication No. WO2014/185517). According to this method, cells usually undergo rapid freezing or slow freezing in a preserving liquid (PCT Patent Publication No. WO2014/185517, Japanese Patent Laid-open No. 2010213692, JPT2012533620, PCT Patent Publication No. WO2005/045007, Japanese Patent Laid-open No. 2007161307, Japanese Patent Laid-open No. 2002204690, Japanese Patent Laid-open No. 2011115058, Japanese Patent Laid-open No. 200393044, JPT1993507715, Japanese Patent Laid-open No. 2004254597, and Japanese Patent Laid-open No. 1996308555). The thus frozen cells scarcely involve difficulties in transportation unlike the sheet-shaped cell culture. However, nothing has been reported concerning freezing and thawing suitable for induced pluripotent stem cells (Japanese Patent Laid-open No. 2010213692) and embryonal stem cells (PCT Patent Publication No. WO2005/045007) as the objects for freezing.

### SUMMARY

The present inventors have been working on the production of a sheet-shaped cell culture of cells derived from pluripotent stem cells or mesenchymal stem cells derived from adipose tissue or bone marrow, which would be expected to be a new cell source, and found in the course of their work that the cells differentiated from these cells poses problems that the cells are highly damaged by freezing, for example, the cells cannot maintain their function and the viability of the cells are decreased after freezing and thawing, and further that the residual undifferentiated pluripotent stem cells may cause of tumorigenic transformation of the transplanted tissue. These findings made the present inventors lead to the recognition that it would be impossible to produce the sheet-shaped cell culture mentioned above unless the foregoing problems are solved. In accordance with an exemplary embodiment, a method is disclosed for cryopreservation of cardiocytes derived from pluripotent stem cells or mesenchymal stem cells derived from adipose tissue or bone marrow, which is free of the foregoing problems, thereby providing a satisfactory sheet-shaped cell culture containing the cardiocytes.

In order to solve the foregoing problems, the present inventors conducted a series of researches and surprisingly found that dissociating a cell population which has been induced to differentiation from pluripotent stem cells or mesenchymal stem cells derived from adipose tissue or bone marrow into cardiocytes to cells and then freezing the cells results in the cardiocytes derived from differentiated pluripotent stem cells or mesenchymal stem cells derived from adipose tissue or bone marrow to maintain their function and the tumorigenicity of the undifferentiated pluripotent stem cells or mesenchymal stem cells derived from adipose tissue or bone marrow to be decreased. These findings have led to the present disclosure.

Accordingly, the present disclosure relates to the followings.

<1> A method for cryopreservation of cardiocytes derived from pluripotent stem cells or mesenchymal stem cells derived from adipose tissue or bone marrow, the method including: a step of dissociating a cell population which has been induced to differentiate into cardiocytes from pluripotent stem cells or mesenchymal stem cells derived from adipose tissue or bone marrow to cells.

<2> The method for cryopreservation according to <1> above which further includes a step of freezing the dissociated cells in a cryopreservation solution which contains a cryoprotectant.

<3> The method for cryopreservation according to <2> above in which the cryoprotectant is one capable of permeation through cell membrane.

<4> The method for cryopreservation according to <2> or <3> above in which the cryoprotectant is one or two or more species selected from the group consisting of dimethyl sulfoxide, ethylene glycol (EG), Propylene glycol (PG), 1,2propanediol (1,2PD), 1,3propanediol (1,3PD), butylene glycol (BG), isopropylene glycol (IPG), dipropylene glycol (DPG), and glycerin.

<5> The method for cryopreservation according to <4> above in which the cryoprotectant is dimethyl sulfoxide.

<6> The method for cryopreservation according to <4> above in which the cryoprotectant is 1,2propanediol.

<7> The method for cryopreservation according to any one of <1> to <6> above in which the pluripotent stem cells are induced pluripotent stem cells.

<8> A method for producing a sheet-shaped cell culture which includes: a step of thawing the frozen cells which are obtained by any one method defined in any one of paragraphs <1> to <7> above; and a step of forming the thawed cells into a sheet-shaped cell culture.

<9> Use of the sheet-shaped cell culture produced by the method according to claim 8, a composition comprising the sheet-shaped cell culture or a kit comprising the frozen cells obtained by the method according to any one of <1> to <7> above, a cell culture solution and a cell culture medium for drug screening.

<10> Use according to <9> above wherein the kit further comprises a medical adhesive and a cell washing solution.

<11> A method for treating a disease in a patient comprising applying an effective amount of the sheet-shaped cell culture produced by the method according to <8> or a composition containing the sheet-shaped cell culture to the patient in need thereof.

<12> A method for increasing the purity of differentiated cardiocytes derived from pluripotent stem cells or mesenchymal stem cells derived from adipose tissue or bone marrow in the cells dissociated from the cell population which has been induced to differentiate into cardiocytes from pluripotent stem cells or mesenchymal stem cells derived from adipose tissue or bone marrow, the method including: a step of freezing the dissociated cells in a cryopreserving solution containing a cryoprotectant.

<13> A method for decreasing the ratio of undifferentiated pluripotent stem cells or mesenchymal stem cells derived from adipose tissue or bone marrow in the cells dissociated from the cell population which has been induced to differentiate into cardiocytes from pluripotent stem cells or mesenchymal stem cells derived from adipose tissue or bone marrow, the method including: a step of freezing the dissociated cells in a cryopreserving solution containing a cryoprotectant.

The method of the present disclosure makes it possible to cryopreserve cells while keeping high viability and maintaining autonomous pulsatility of cardiocytes derived from pluripotent stem cells or mesenchymal stem cells derived from adipose tissue or bone marrow by dissociating a cell population which has been induced to differentiate into cardiocytes from pluripotent stem cells or mesenchymal stem cells derived from adipose tissue or bone marrow to the cells. Further, the method of the present disclosure also can reduce pluripotency and proliferative property of the residual pluripotent stem cells or mesenchymal stem cells derived from adipose tissue or bone marrow after induction into differentiation, which would be a cause of tumorigenesis in clinical application. In addition, it is possible to produce a sheet-shaped cell culture which maintains the cell viability and autonomous pulsatility from the cardiocytes obtained by the method of the present disclosure derived from the cryopreserved pluripotent stem cells or mesenchymal stem cells derived from adipose tissue or bone marrow. The freeze-thaw process of the method of the present disclosure is compatible with the conventional method for producing a sheet-shaped cell culture and is fuss-free and low cost, thus, the method of the present disclosure can be used within a broad range for production of sheet-shaped cell cultures.

### BRIEF DESCRIPTION OF THE DRAWINGS

FIG. 1 is a graph depicting the ratio of cells positive to SSEA-4 and the ratio of cells positive to c-TNT, which have been observed before and after the freezing of a cell population containing cardiocytes derived from iPS cells.

FIG. 2 is a graph depicting the ratio of cells positive to c-TNT which has been observed before and after the freezing of a cell population containing cardiocytes derived from iPS cells.

FIG. 3 is a graph depicting the ratio of cells positive to Tra-1-60 and the ratio of cells positive to c-TNT which have been observed before and after the freezing of a cell population containing cardiocytes derived from iPS cells.

FIG. 4 is a graph depicting the ratio of cells positive to SSEA-4 which has been observed before and after the freezing of iPS cells.

FIG. 5 is a photograph depicting the external appearance of the completed sheet-shaped cell culture.

FIG. 6 is an optical microscopic photograph depicting the completed sheet-shaped cell culture, with cells partly stained with hematoxylin-eosin.

FIG. 7 is a fluorescent microscopic photograph depicting the completed sheet-shaped cell culture, with cells partly undergone multiple labeling.

FIG. 8 is a graph depicting the autonomous synchronous pulsation observed in the completed sheet-shaped cell culture.

FIG. 9 is a graph depicting the stability of cardiocytes derived from iPS cells.

FIG. 10 is a graph depicting the effect of freezing that depends on the cryopreserving liquid used.

FIG. 11 is a graph depicting the effect of freezing that depends on the freezing method used.

FIG. 12 is a graph depicting the effectiveness of the sheet of cardiocytes derived from iPS cells.

### DETAILED DESCRIPTION

The technical terms and scientific terms used in this specification have the same meanings as understood by those who are skilled in the art, unless otherwise defined herein. All of the patents, patent applications, and other publications (including information available from Internet) cited in this specification are incorporated herein by reference in its entirety.

In accordance with an exemplary embodiment, a method is disclosed for cryopreservation of cardiocytes derived from pluripotent stem cells or mesenchymal stem cells derived from adipose tissue or bone marrow, the method comprising a step of dissociating a cell population which has been induced to differentiate into cardiocytes from pluripotent stem cells or mesenchymal stem cells derived from adipose tissue or bone marrow to cells. While not wishing to be bound by any particular theory, it is considered that, by freezing the cells that are dissociated from the cell population which has been induced to differentiate into cardiocytes from pluripotent stem cells or mesenchymal stem cells derived from adipose tissue or bone marrow, cardiocytes derived from pluripotent stem cells or mesenchymal stem cells derived from adipose tissue or bone marrow remain with maintaining their autonomous pulsatility, as well as eliminating the residual undifferentiated pluripotent stem cells or mesenchymal stem cells derived from adipose tissue or bone marrow after induction to differentiation.

The term “pluripotent stem cells” used herein is known in the technical field concerned; it denotes cells capable of differentiation into various tissues in a living body. Non-limiting examples of the pluripotent stem cells include embryonal stem cells (ES cells), nuclear transfer embryonal stem cells (ntES cells), and induced pluripotent stem cells (iPS cells).

The term “mesenchymal stem cells” is known in the technical field concerned; it denotes cells which are present in the mesenchymal tissue and capable of differentiation into cells belonging to the mesenchymal tissue. In this specification, mesenchymal stem cell denotes, unless otherwise mentioned, mesenchymal stem cell derived from adipose tissue or bone marrow.

In this specification, the term “cardiocytes derived from pluripotent stem cells or mesenchymal stem cells derived from adipose tissue or bone marrow” means any cells derived from pluripotent stem cells or mesenchymal stem cells derived from adipose tissue or bone marrow, that have the features of cardiocytes. Non-limiting examples of the features of cardiocytes include the expression of cardiocyte marker or the presence of autonomous pulsation. Non-limiting examples of the cardiocyte marker include cTNT (cardiac troponin T), CD172a (also known as SIRPA or SHPS1), KDR (also known as CD309, FLK1, or VEGFR2), PDGFRA, EMILIN2, and VCAM. In one embodiment, the cardiocytes derived from pluripotent stem cells or mesenchymal stem cells are positive to cTNT and/or positive to CD172a.

In this specification, the term “a cell population which has been induced to differentiate into cardiocytes from pluripotent stem cells or mesenchymal stem cells derived from adipose tissue or bone marrow” means a mass of cells or an aggregate of cells which contains cardiocytes obtained by culture and induction from pluripotent stem cells or mesenchymal stem cells derived from adipose tissue or bone marrow and undifferentiated pluripotent stem cells and/or mesenchymal stem cells derived from adipose tissue or bone marrow. The cell population may also contain other induced cell species. The cell population can be obtained according to the method for forming embryoids (as disclosed by Burridge et al., Cell Stem Cell. 2012 Jan. 6; 10(1): 1628), by induction of cardiocytes from pluripotent stem cells or mesenchymal stem cells, for example, subjecting induces pluripotent stem cells or mesenchymal stem cells to differentiation into cardiocytes. In the foregoing method, efficient of induction can be increased by sequential affection with mesoderm inducing factor (such as activin A, BMP4, bFGF, VEGF, and SCF), cardiac specification factor (such as VEGF, DKK1, Wnt signal inhibitor (for example, IWR1, IWP2, and IWP4), BMP signal inhibitor (for example, NOGGIN), TGFβ/activin/NODAL signal inhibitor (for example, SB431542), retinoic acid signal inhibitor, and cardiac differentiation factor (such as VEGF, bFGF, and DKK1).) In one embodiment, the treatment for induction of pluripotent stem cells into cardiocytes includes sequential actions on the cell population resulting from suspension culture with (1) BMP4, (2) combination of BMP4, bFGF, and activin A, (3) IWR1, and (4) combination of VEGF and bFGF.

In accordance with an exemplary embodiment, the method according to the present disclosure includes the step of dissociating a cell population to cells can be accomplished by any known technique, the non-limiting examples of which include chemical processes to perform cell dissociation with the help of dissociating agent such as trypsin, ethylenediaminetetraacetic acid (EDTA), pronase, dispase, collagenase, and CTK (from ReproCELL Inc.) and also include physical processes such as pipetting. Cell dissociation may also be accomplished by adhesion culture, with cell population adhering to the culture substrate.

In one aspect of the present disclosure, the method for cryopreservation further includes the step of freezing dissociated cells in a cryopreserving solution which contains a cryoprotectant. This freezing step may be accomplished by any known technique. Non-limiting example of such technique includes applying cells in a container to a freezing means such as a freezer, a deep freezer, or a low-temperature medium (such as liquid nitrogen.) The freezing temperature is not specifically limited so long as it is low enough to partly, preferably entirely freeze the cell population in a container. In accordance with an exemplary embodiment, the freezing temperature should be equal to or lower than 0° C., preferably equal to or lower than −20° C., more preferably equal to or lower than −40° C., and still more preferably equal to or lower than −80° C. The freezing operation may be accomplished at any cooling rate without particular limitations so long as it does not impair considerably the survival ratio or function of the cells after thawing, a typical cooling rate would be 1 to 5 hours, preferably 2 to 4 hours, particularly about 3 hours for cooling from 4° C. to −80° C. In accordance with an exemplary embodiment, an example of the cooling rate is 0.46° C./minute. This cooling rate can be achieved by placing a container containing cells directly or with housed in a freezing vessel in freezing means which is set at a desired temperature. The freezing vessel may have a function to control the temperature in it to decrease at a prescribed cooling rate. Any known freezing vessel, for example BICELL® (Nippon Freezer), can be used. In addition, the foregoing cooling rate may be achieved by using a freezer or deep freezer capable of programming to control the cooling rate. Any known freezers or deep freezers including program freezers, such as PDF2000G (STREX Inc.) and KRYO56016 (ASAHI LIFE SCIENCE Co., Ltd.) can be used.

The freezing operation may be accomplished by using, as the cryopreserving solution, a culture medium or a physiological buffer solution containing cells immersed therein and adding cryoprotectant to them, or replacing a culture medium with a cryopreserving solution containing a cryoprotectant. Thus, the method of the present disclosure may further comprise a step of adding a cryoprotectant to the culture medium or a step of replacing the culture medium with a cryopreserving solution. The latter case may be performed by removing the culture medium substantially entirely and then adding a cryopreserving solution or by adding a cryopreserving solution while partly leaving the culture medium, so long as the solution in which cells are immersed contains a cryoprotective solution in an effective concentration at the time of freezing. The term “effective concentration” used herein means that the cryoprotectant exists in an amount sufficient to produce the cryoprotective effect without toxicity. The cryoprotective effect means an ability to prevent the cells after thawing from decreasing in survival ratio, vitality, and functions compared with the case free from the cryoprotectant. Such a concentration is known to those who are skilled in the art or may be appropriately determined by routine experiments.

The method according to the present disclosure may employ any cryoprotectant without specific restrictions so long as it is capable of permeation through cell membrane. The cryoprotectants can include, for example, dimethyl sulfoxide (DMSO), ethylene glycol (EG), propylene glycol (PG), 1,2propanediol (1,2PD), 1,3propanediol (1,3PD), butylene glycol (BG), isopropylene glycol (IPG), dipropylene glycol (DPG), and glycerin. Among these, DMSO and 1,2PD are particularly preferable. These cryoprotectants may be used alone or in combination with one another.

The cryoprotectant may be used in combination with any extracellular cryoprotectant, such as polyethylene glycol, carboxymethyl cellulose sodium, polyvinylpyrrolidone, hydroxyethyl starch (HES), dextran, and albumin.

In accordance with an exemplary embodiment, the cryoprotectant should be added to the culture medium or the cryopreserving solution in any amount (concentration) without specific restrictions so long as it is the effective concentration defined above. Typical concentrations based on the culture medium or cryopreserving solution is 2% to 20% (v/v), preferably 5% to 15%, more preferably 8% to 12%, and most desirably 10%. These values of concentration are not necessarily mandatory; it is possible to use any values outside the foregoing values if such values are known for each cryoprotectant or experimentally determined, and such values are covered by the scope of the present disclosure. For example, the adequate concentration of DMSO (based on the total amount of culture medium or cryopreserving solution) is 2% to 20% (v/v), preferably 2.5% to 12.5%, and most desirably 5% to 10%.

According to the present disclosure, the cardiocytes derived from pluripotent stem cells or mesenchymal stem cells can be purified after induction. Purification may be achieved in various ways as exemplified below. Separation with the help of markers (such as cell surface markers) specific to cardiocytes, which includes magnetic cell separation method (MACS), flow cytometry method, and affinity separation method. Use of the specific promoter which causes selective markers (genes resistant to antibiotics) to express. Use of the property of cardiocytes demanding nutrition, such as a method for getting rid of other cells than cardiocytes by performing cell culture on a culture medium free of nutrients necessary for survival of other cells than cardiocytes (Japanese Patent Laid-open No. 2013143968.) Selection of cells capable of survival under poor nutritional conditions (WO 2007/088874.) Recovery of cardiocytes by means of difference in adhesion to a culture substrate coated with adhesive protein between cardiocytes and other cells than cardiocytes (Japanese Patent Application No. 2014188180.) Combination of the foregoing methods (see Burridge et al. mentioned above.) In accordance with an exemplary embodiment, the cell surface marker specific to cardiocytes may be exemplified by CD172a, KDR, PDGFRA, EMILIN2, and VCAM. The promoter specific to cardiocytes may be exemplified by NKX2-5, MYH6, MLC2V, and ISL1. In one embodiment, the purification of cardiocytes is accomplished with the help of the cell surface marker identified as CD172a.

The cell population containing cardiocytes derived from pluripotent stem cells or mesenchymal stem cells may be freed of some of them which have not yet differentiated. Undifferentiated cells remaining in the cell population containing cardiocytes derived from pluripotent stem cells or mesenchymal stem cells may suffer from canceration after transplantation. Removal of undifferentiated cells may be accomplished by any known method. Purification may be achieved in various ways as exemplified below. Separation with the help of markers (such as cell surface markers) specific to undifferentiated cells, which includes magnetic cell separation method (MACS), flow cytometry method, and affinity separation method. Use of the specific promoter which causes selective markers (genes resistant to antibiotics) to express. Getting rid of undifferentiated cells by performing cell culture on a culture medium free of nutrients (such as methionine) necessary for survival of undifferentiated cells. Treatment with a medicine targeting the surface antigen of undifferentiated cells.

The known methods for removing undifferentiated cells include those disclosed in the following patents and documents such as WO2014/12646, WO2012/056997, WO2012/147992, WO2012/133674, WO2012/012803 (JPT2013/535194), WO2012/078153 (JPT2014501518), Japanese Patent Laid-open No. 2013143968, Cell Stem Cell vol. 12 Jan. 2013, page 127137, and PNAS 2013 Aug. 27; 110(35): E328190.

In accordance with an exemplary embodiment, the cardiocytes derived from pluripotent stem cells or mesenchymal stem cells may be in the form of cell population of cardiocytes which has been obtained from pluripotent stem cells or mesenchymal stem cells and subsequently purified as mentioned above. The cell population of cardiocytes may have a purity, for example, as follows in terms of the ratio of the number of cells positive to the cardiocyte marker to the total number of cells in the cell population of cardiocytes: higher than approximately 85%, higher than approximately 86%, higher than approximately 87%, higher than approximately 88%, higher than approximately 89%, higher than approximately 90%, higher than approximately 91%, higher than approximately 92%, higher than approximately 93%, higher than approximately 94%, higher than approximately 95%, higher than approximately 96%, higher than approximately 97%, higher than approximately 98%, and higher than approximately 99%. In one embodiment of the present disclosure, the cardiocytes derived from pluripotent stem cells or mesenchymal stem cells take on the form of cell population in which the purity of cardiocytes exceeds 90%.

The cell population containing the cardiocytes derived from pluripotent stem cells or mesenchymal stem cells may be any of the following. The one which is obtained by induction into cardiocytes which follows the treatment of pluripotent stem cells or mesenchymal stem cells for induction to cardiocytes. The one which is obtained by purification of cardiocytes that follows induction into cardiocytes. The one which is made impure by partial removal of cardiocytes from the cell population after induction into cardiocytes. The one which is a mixture of the cell population of purified cardiocytes and another cell population. In one embodiment of the present disclosure, the cell population containing the cardiocytes derived from pluripotent stem cells or mesenchymal stem cells may be a mixture of two cell populations, the one being a cell population of cardiocytes obtained by purification of the cell population that results from pluripotent stem cells or mesenchymal stem cells after induction into cardiocytes, and the other being a cell population of noncardiocytes remaining after purification.

Another aspect of the present disclosure relates to a method for producing a sheet-shaped cell culture which includes a step of thawing the frozen cells obtained by the foregoing method and a step of forming a sheet-shaped cell culture.

The term “sheet-shaped cell culture” used in the present disclosure denotes something forming a sheet in which cells are jointed each other. The cells may be jointed directly (including jointed through cell elements such as adhesion molecule) and/or jointed indirectly through intervening substance. The intervening substance is not specifically limited so long as it is capable of physically (or mechanically) join cells together. The intervening substance, can include, for example, extracellular matrix, and should preferably be one which is derived from cells, particularly cells constituting the cell culture. The physical (or mechanical) joining of cells may be enhanced functionally (for example, chemically or electrically.) The sheet-shaped cell culture may be a monolayer one (composed of one cell layer) or a multilayer one (composed of two or more cell layers.) The one composed of two layers, three layers, four layers, five layers, or six layers may be acceptable.

In accordance with an exemplary embodiment, the sheet-shaped cell culture should preferably be one, which does not contain any scaffold (support.) It is known that a scaffold is commonly used in the technical field of the present disclosure in order that the sheet-like cell culture physically maintains its integrity by means of cells attached to the surface or inside thereof. Known examples of such a scaffold include a film of polyvinylidene difluoride (PVDF.) In the present disclosure, however, such a scaffold is not necessary for the sheet-shaped cell culture, which maintains its physical integrity without any scaffold. In addition, the sheet-shaped cell culture should preferably be composed solely of the substance derived from cells constituting the cell culture medium, with any other impurities excluded.

The sheet-shaped cell culture should be constituted of cells derived from any living organisms capable of receiving treatment with the sheet-shaped cell culture. Such living organisms non-limitingly can include human, primate (excluding human), dog, cat, pig, horse, goat, sheep, rodent (for example, mouse, rat, hamster, and guinea pig), and rabbit. In one embodiment of the present disclosure, the sheet-shaped cell culture should be constructed of human cells.

The sheet-shaped cell culture should be constituted of cells derived from heterogeneous cells or homogeneous cells. The term “cells derived from heterogeneous cells” denotes those cells derived from a living organism different in species from the recipient to which the sheet-shaped cell culture is transplanted. For example, “cells derived from heterogeneous cells” denotes those cells derived from a monkey or pig if the recipient is a human. The term “cells derived from homogeneous cells” denotes those cells derived from a living organism same in species with the recipient. For example, the term “cells derived from homogeneous cells” denotes those cells derived from a human if the recipient is a human. The term the cells derived from homogeneous cells include those cells derived from autonomous cells, or those cells derived from the recipient and those cells derived from homogenous and nonautonomous cells. Those cells derived from the autonomous cells are preferable in the present disclosure because they do not cause rejection. However, it is also possible to use those cells derived from heterogeneous cells and homogenous and nonautonomous cells. In this case, those cells may need immune suppression treatment in order to suppress the rejection. In this specification, the term “cells derived from nonautonomous cells” generally denotes cells derived from cells excluding autonomous cells, namely cells derived from heterogeneous cells and cells derived from homogenous nonautonomous cells. In one embodiment according to the present disclosure, the cells are autonomous cells or nonautonomous cells. In another embodiment according to the present disclosure, the cells are autonomous cells. In further another embodiment according to the present disclosure, the cells are nonautonomous cells.

According to the present disclosure, no restrictions are imposed on the autonomous or nonautonomous pluripotent stem cells. They may be obtained from collected autonomous or nonautonomous cells including skin cells (such as fibroblast and keratinocyte) and blood cells (such as peripheral blood mononuclear cells) by introduction of genes such as OCT3/4, SOX2, KLF4, and CMYC for induction into autonomous or nonautonomous iPS cells. The method for induction from somatic cells into iPS cells is known in the technical field concerned. (See Bayart and CohenHaguenauer, Curr Gene Ther. 2013 April; 13(2): 7392, for example.)

In accordance with an exemplary embodiment, the method according to the present disclosure includes a step of thawing frozen cells. This step may be accomplished by any known technique, which can include heating frozen cells to a temperature higher than the freezing temperature. Such heating may be achieved by using a medium (in the form of solid, liquid, or gas) held in water, a water bath, incubator, thermostat, or the like. Thawing may also be achieved by dipping frozen cells in a medium (such as culture medium) kept at a temperature higher than the freezing temperature. There are any other techniques. The means for thawing or the medium for dipping may be set at any temperature high enough for frozen cells to thaw within a prescribed period of time. In accordance with an exemplary embodiment, the thawing temperature can be 4° C. to 50° C., preferably 30° C. to 40° C., and more preferably 36° C. to 38° C. The duration for thawing is not specifically limited so long as it does not adversely affect the survival ratio and functions of cells after thawing. In accordance with an exemplary embodiment, the duration for thawing can be typically within two minutes, particularly within 20 seconds. This limited length of duration significantly saves the survival ratio. Thawing may be achieved within a certain length of time which should be appropriately adjusted according to the thawing means, the temperature of the dipping medium, and the volume or composition of the culture medium or cryopreserving solution used at the time of freezing.

In accordance with an exemplary embodiment, the method according to the present disclosure may include a step of washing cells which follows the step of thawing frozen cells and precedes the step of forming the sheet-shaped cell culture. The washing of cells may be accomplished by any known technique (without restrictions), which typically includes of suspending cells in a cell washing solution, separating cells by centrifuging, discarding supernatant solution, and recovering precipitated cells. The cell washing solution may be a culture medium or physiological buffer solution containing or not containing serum or serum components (such as serum albumin.) The step of washing cells may include suspension, centrifugal separation, and recovery, which may be performed once or more than twice, up to five times. In one embodiment of the present disclosure, the step of washing cells may be performed immediately after the step of thawing frozen cells. The washing of cells may be accomplished by using any commercial cell washing solution, an example of which is available under a trade name of Cellotion (from Nippon Zenyaku Kogyo Co., Ltd.)

The method according to the present disclosure includes a step of forming the sheet-shaped cell culture. This step may be accomplished by any known technique without restrictions, such as the one disclosed in JPT2007528755 and Japanese Patent Laid-open No. 2012115254.

One embodiment of the present disclosure includes a step of forming the sheet-shaped cell culture, the step including a step of inoculating cells on a culture medium and a step for the cells to grow into a sheet form.

One embodiment of the present disclosure also includes a step of culturing coated cells (which result from coating the entire cell surface with an adhesive film.) The step of culturing coated cells may employ coated cells and cultured cells, which are bonded together through an adhesive layer. The adhesive film covering the coated cells is not specifically limited so long as it is capable of bonding cultured cells together. It should preferably be a natural polymer (such as protein) or a chemically synthesized polymer having a molecular weight ranging, for example, from 1,000 to 10,000,000. In addition, the adhesive film should preferably be a laminate film composed of a first layer containing a first substance and a second layer containing a second substance differing from the first substance. The combination of a first substance and a second substance should preferably be one which is composed of a polymer containing the arrangement of arginine-glycine-aspartic acid to which integrin bonds and a polymer containing the RGD arrangement, the polymer reacting each other. The polymer containing the RGD arrangement may be a protein originally having the RGD arrangement. The polymer which mutually reacts with the polymer containing the RGD arrangement contains water-soluble proteins, such as collagen, gelatin, proteoglycan, integrin, enzyme, and antibody. The abovementioned step causes the adhesion film formed on individual cells to mutually react to form a tissue, and eventually it forms the sheet-shaped cell culture having a three-dimensional structure.

The culture substrate is not specifically limited so long as it permits cells to form a cell culture thereon. The culture substrate can include, for example, a solid or semisolid surface of a container made of various materials. The container should preferably be one made of a material impermeable to liquid such as culture medium. Such a material nonlimitingly includes polyethylene, polypropylene, Teflon (registered trademark), polyethylene terephthalate, polymethyl methacrylate, nylon6,6, polyvinyl alcohol, cellulose, silicone, polystyrene, glass, polyacrylamide, polydimethylacrylamide, and metal (such as iron, stainless steel, aluminum, copper, and brass). In accordance with an exemplary embodiment, the container should preferably be one which has at least one flat surface. Such a container nonlimitingly includes cell culture dishes and cell culture bottles. In addition, the container may have a solid or semisolid surface. The solid surface may be the surface of plate or container made of the foregoing materials. The semisolid surface may be the surface of gel, soft polymer matrix, or film. The culture substrate may be prepared from the foregoing materials or purchased from a commercial source. The preferable culture substrate nonlimitingly includes one which has an adhesive surface that permits the sheet-shaped cell culture to be formed thereon. Typical examples of the substrate are listed below. Substrate having a hydrophilic surface, such as corona discharge treated polystyrene. Substrate coated with a hydrophilic compound such as collagen gel and hydrophilic polymer. Substrate coated with extracellular matrix or cell adhesive factor, the former including collagen, fibronectin, laminin, vitronectin, proteoglycan, and glycosaminoglycan, and the latter including cadherin family, selectin family, and integrin family. The substrates listed above are commercially available (for example, Corning® TCTreated Culture Dish, made by Corning Inc.).

The culture substrate may be coated with any material which changes in physical properties in response to stimuli such as temperature and light. Non-limiting examples of such a material include the following known materials: (Meth)acrylamide compound, N-alkylsubstituted (meth)acrylamide derivatives (for example, N-ethylacrylamide, N-propylacrylamide, N-propylmethacrylamide, N-isopropylacrylamide, N-isopropylmethacrylamide, N-cyclopropylacrylamide, N-cyclopropylmethacrylamide, N-ethoxyethylacrylamide, N-ethoxyethylmethacrylamide, N-tetrahydrofurfurylacrylamide, and N-tetrahydrofurfurylmethacrylamide), N,N-dialkylsubstituted (meth)acrylamide derivatives (for example, N,N-dimethyl(meth)acrylamide, N,N-ethylmethylacrylamide, and N,N-diethylacrylamide), (Meth)acrylamide derivative having cyclic groups (for example, 1(1oxo2propenyl) pyrrolidine, 1(1oxo2propenyl)piperidine, 4(1oxo2propenyl)morpholine, 1(1oxo2methyl2propenyl)pyrrolidine, 1(1oxo2methyl2propenyl)piperidine, and 4(1oxo2methyl2propenyl)morpholine), or a temperature responsive material made from homopolymer or copolymer of vinyl ether derivative (such as methyl vinyl ether), a light absorptive polymer having an azobenzene group, a copolymer composed of a vinyl derivative of triphenylmethaneleucohydroxide and an acrylamide-related monomer, a light responsive material such as N-isopropylacrylamide gel containing spirobenzopyran (see, for example, Japanese Patent Laid-open Nos. 1990211865 and 200333177). When properly stimulated, the foregoing materials change their physical properties such as hydrophilic nature or hydrophobic nature, thereby abrasion of the cell culture attached to the materials would be facilitated. In addition, commercial culture dishes coated with a temperature responsive material, under a trade name of UpCell® from CellSeed Inc. may be used for the method according to the present disclosure.

In accordance with an exemplary embodiment, the foregoing culture substrate should preferably be flat although it may have various shapes. It is not specifically limited in area. However, an exemplary area is, for example, 1 cm² to 200 cm², preferably 2 cm² to 100 cm², and more preferably 3 cm² to 50 cm².

The culture substrate is inoculated with cells by any known technique under any known conditions. For example, the inoculation may be accomplished by loading the culture container with the culture medium in which cells are suspended. This step may be facilitated by using a dropper, pipette, or the like.

In one preferred exemplary embodiment of the present disclosure, the inoculation is performed in such a way that the cell suspension contains as many cells as necessary to form the sheet-shaped cell culture after cultivation, for example, for 1 day to 7 days. The number of cells per unit area for inoculation should, for example, be 5×10⁴/cm² to 5×10⁶/cm², preferably 1×10⁵/cm² to 2×10⁶/cm², and more preferably 1×10⁵/cm² to 1×10⁶/cm².

The thus inoculated cells are made into a sheet by any known technique under any known conditions without specific restrictions, as disclosed in JPT2007528755, for example. It is believed that the cells assume a sheet-like shape as the result of cells adhering to one another with adhering molecules or extracellular matrix interposed between them (which function as the cell bonding mechanism.) Consequently, the step of allowing inoculated cells to assume a sheet-like shape is accomplished by culturing cells under a condition suitable for intercellular adhesion. The condition is not specifically limited but is similar to the one ordinarily employed for cell culture. In accordance with an exemplary embodiment, a typical condition for cell culture is 37° C. and 5% CO₂. Culture may be performed under normal pressure (atmospheric pressure.) An optimal condition may be established by those skilled in the art. In this specification, the term “sheet forming culture” may be used to denote culture intended to turn inoculated cells into a sheet-like shape.

In one embodiment of the present disclosure, cell culture may be carried out for a prescribed period of time, such as, for example, less than 7 days, preferably less than 5 days, and more preferably less than 3 days.

Cell culture employs a cell culture medium (occasionally abbreviated as “culture medium” or “medium” hereinafter), which is not specifically limited so long as it ensures cell survival. It typically includes those composed mainly of amino acids, vitamins, and electrolytes. In one embodiment of the present disclosure, the culture medium is one which is based on a basic culture medium for cell culture. The basic culture medium is not specifically limited but includes those which are known as DMEM, MEM, F12, DME, RPMI11640, MCDB (such as MCDB102, 104, 107, 120, 131, 153, and 199), L15, SkBM, and RITC807. They are mostly commercially available and their compositions are known.

The basic culture medium may be used as purchased (in its original form) or after modification suitable for cell species and cell conditions. The basic culture medium used in the present disclosure is not limited in composition to the known ones, but it may be modified by adding, removing, increasing, or reducing one or more than two components.

In accordance with an exemplary embodiment, the basic culture medium contains amino acids, vitamins, and electrolytes listed below without specific restrictions. Examples of amino acids include: L-arginine, L-cystine, L-glutamine, glycine, L-histidine, L-isoleucine, L-leucine, L-lysine, L-methionine, L-phenylalanine, L-serine, L-threonine, L-tryptophan, L-tyrosine, and L-valine. Examples of vitamins include: calcium D-pantothenate, choline chloride, folic acid, i-Inositol, niacinamide, riboflavin, thiamin, pyridoxine, biotin, lipoic acid, vitamin B12, adenine, and thymidine. Examples of electrolytes include: CaCl₂, KCl, MgSO₄, NaCl, NaH₂PO₄, NaHCO₃, Fe(NO₃)₃, FeSO₄, CuSO₄, MnSO₄, Na₂SiO₃, (NH₄)₆Mo₇O₂₄, NaVO₃, NiCl₂, and ZnSO₄. The basic culture medium may further contain saccharidies (such as D-Glucose), sodium pyruvate, pH indicator (such as Phenol Red), and putrescine.

In one exemplary embodiment of the present disclosure, the basic medium may contain amino acids in concentrations exemplified below. L-arginine: 63.2 mg/L to 84 mg/L, L-cystine: 35 mg/L to 63 mg/L, L-glutamine: 4.4 mg/L to 584 mg/L, Glycine: 2.3 mg/L to 30 mg/L, L-histidine: 42 mg/L, L-isoleucine: 66 mg/L to 105 mg/L, L-leucine: 105 mg/L to 131 mg/L, L-lysine: 146 mg/L to 182 mg/L, L-methionine: 15 mg/L to 30 mg/L, L-phenylalanine: 33 mg/L to 66 mg/L, L-serine: 32 mg/L to 42 mg/L, L-threonine: 12 mg/L to 95 mg/L, L-tryptophan: 4.1 mg/L to 16 mg/L, L-tyrosine: 18.1 mg/L to 104 mg/L, L-valine: 94 mg/L to 117 mg/L.

In one exemplary embodiment of the present disclosure, the basic medium may contain vitamins in concentrations exemplified below. D calcium pantothenate: 4 mg/L to 12 mg/L, choline chloride: 4 mg/L to 14 mg/L, folic acid: 0.6 mg/L to 4 mg/L, i-Inositol: 7.2 mg/L, niacinamide: 4 mg/L to 6.1 mg/L, riboflavin: 0.0038 mg/L to 0.4 mg/L, thiamin: 3.4 mg/L to 4 mg/L, pyridoxine: 2.1 mg/L to 4 mg/L.

In accordance with an exemplary embodiment, the cell culture medium may contain, in addition to the foregoing components, one or two or more species of additives, such as serum, growth factor, steroid, and selenium. However, they should preferably be excluded at the time of clinical application because they could cause side effects (such as anaphylaxis shock) to the recipient. (They are inevitable impurities resulting from the manufacturing process.) Therefore, according to a preferable embodiment of the present disclosure, the cell culture medium should not contain even one species of the additives in an effective amount. According to a more preferable embodiment of the present disclosure, the cell culture medium should be substantially free of the additives. According to a particularly preferable embodiment of the present disclosure, the cell culture medium should be composed solely of the basic medium and substantially free of the additives.

According to another embodiment of the present disclosure, the cell culture medium may contain ROCK (Rhoassociated coiled-coil forming kinase) inhibitor Y-27632.

An example of the cell culture medium that can be used in the present disclosure is 20% FBSDMEM/F12.

In one embodiment of the present disclosure, the step of forming the sheet-shaped cell culture may include a substep for purification of cardiocytes and removal of undifferentiated cells. This substep is not specifically limited so long as it can be performed under the following conditions simultaneously with the formation of the sheet-shaped cell culture. Low serum content, low saccharide content, low nutrient content, low calcium content, weak acid pH, lactic acid added, aspartic acid added, glutamic acid added, and/or pyruvic acid added. Additionally or alternatively, the substep may be performed in the cell culture medium free of at least one amino acid selected from the group consisting of methionine, leucine, cysteine, tyrosine, and arginine. (These conditions are disclosed in Japanese Patent Laid-open No. 2013143968 and WO2012/056997). Condition of low serum content denotes the condition in which serum is absent or present such that the serum content is 0% to 10% of the total content (100%) of the serum or serum component or artificial physiologically active substance, which has been added to the cell culture medium used for induction to differentiation. Condition of low saccharide content denotes the condition in which saccharide is absent or the content of saccharide is less than 1% of the saccharide in the cell culture medium used at the time of induction to differentiation. Condition of low nutrient content denotes the condition in which the total content of nutrients to be contained in the cell culture medium is equal to or lower than 10% of the nutrients in the cell culture medium. Condition of low calcium content denotes the condition in which the calcium concentration in the cell culture medium is 0.3 mM to 1.3 mM. Condition of weak acid pH denotes the condition in which the pH of the cell culture medium is 6 to 7. Condition of lactic acid added denotes the condition in which lactic acid is added in an amount of 0.1 mM to 5 mM to the cell culture medium. Condition of aspartic acid added and glutamic acid added denotes the condition in which aspartic acid and glutamic acid are added in an amount of 20 mg/L to 100 mg/L each. Conditions of pyruvic acid added denotes the condition in which pyruvic acid is added to the cell culture medium in an amount of 0.5 mM to 5 mM. Condition in which the cell culture medium is free of at least one amino acid selected from the group consisting of methionine, leucine, cysteine, tyrosine, and arginine denotes the condition in which the content of specific amino acid is none or very little in the cell culture medium. The very little amount denotes an amount, for example, equal to or less than 20 μM, preferably equal to or less than 10 μM, more preferably equal to or less than 1 μM, and most desirably equal to or less than 0.1 μM. It is considered that the cultivation under these conditions selectively supplies nutrients to the cardiocytes, which have undergone induction to differentiation, thereby reducing or removing undifferentiated cells and improving the ratio of induction to differentiation and eventually providing pure cardiocytes.

In accordance with an exemplary embodiment, the method according to the present disclosure may include the step of forming the sheet-shaped cell culture and an additional subsequent step of recovering the thus formed sheet-shaped cell culture. The step of recovering the sheet-shaped cell culture is not specifically limited so long as it is capable of peeling (freeing) the sheet-shaped cell culture at least partly from the scaffold while keeping its sheet structure. It may be accomplished by, for example, enzymatic treatment with protease (such as trypsin) and/or mechanical treatment with pipetting. The recovering step may also be performed without resorting to enzyme in the case where the cell culture is formed on a culture substrate coated with any material which changes in properties in response to stimuli (such as temperature and light.)

In accordance with an exemplary embodiment, one embodiment of the method according to the present disclosure may include the step of forming the sheet-shaped cell culture and additional subsequent steps of recovering the thus formed sheet-shaped cell culture and thawing the frozen cells within 48 hours after recovery. The interval between the thawing of the frozen cells and the recovery of the sheet-shaped cell culture, for example, should be equal to or shorter than 48 hours, preferably equal to or shorter than 36 hours, and more preferably equal to or shorter than 24 hours. The shorter is the interval, the more active is the sheet-shaped cell culture.

In accordance with an exemplary embodiment, the method according to the present disclosure may include a step of freezing cells and an additional ensuing step of proliferating cells. The step of proliferating cells may be accomplished by any method for cell culture and proliferation, which is known to those who are skilled in the art. The method according to the present disclosure may be performed in such a way that the sheet-shaped cell culture is formed without substantial cell proliferation after the step of thawing frozen cells or in such a way that the step of thawing frozen cells is performed within 48 hours after the recovery of the sheet-shaped cell culture. In this case, it is useful to obtain the desired number of cells to perform the step of proliferating cells prior to the step of freezing cells.

In accordance with an exemplary embodiment, the method according to the present disclosure may also include an additional step to be performed before or after the step of freezing cells. This additional step includes dispersing the cell population (which has been induced to differentiate into cardiocytes from pluripotent stem cells or mesenchymal stem cells derived from adipose tissue or bone marrow) into separate cells, purifying the cardiocytes, and allowing the cardiocytes to form cell aggregates. The technique of dispersing the cell population into separate cells, thereby purifying the cardiocytes, is not specifically limited so long as it is capable of dispersing cardiocytes into separate cells with the help of enzymatic action, thereby purifying individual cardiocytes. For example, it is possible to select and purify only cardiocytes by using the mitochondria in cardiocytes as the index (as disclosed in WO2006/022377), and it is also possible to select only cells capable of survival under the condition of low nutrient (as disclosed in WO2007/088874.) The technique of allowing the thus purified cardiocytes to form cell aggregates may be performed in any known way. An example of the technique includes culturing the cells on a serum-free medium, thereby causing the cells to form cell aggregates. The medium to be used for this technique should preferably contain at least one substance selected from the group consists insulin (0.1 mg/L to 10 mg/L), transferrin (0.1 μg/L to 10 μg/L), basic fibroblast growth factor (bFGF) (0.1 μg/L to 10 μg/L), epidermal growth factor (1 ng/mL to 1000 ng/mL), growth factor derived from blood plate (1 ng/mL to 1000 ng/mL), and endothelin) (ET1) (1×10⁸ M to 1×10⁶ M.) Other medium compositions and culture conditions than mentioned above will be devised by those who are skilled in the art with reference to WO2009/017254.

In accordance with an exemplary embodiment, one embodiment of the method according to the present disclosure comprises the step of introducing genes into the cells. Another embodiment does not comprise the step of introducing genes into the cells. The genes to be introduced are not specifically limited so long as they are useful for the treatment of target diseases. The examples of the genes may include cytokines such as HGF and VEGF. The introduction of genes may be accomplished by any known methods, such as calcium phosphate method, lipofection method, ultrasonic introduction method, electroporation method, particle gun method, a method using virus vectors such as adenovirus vector and retrovirus vector, and microinjection method. The injection of genes into the cells may be performed at any time without specific restrictions, for example, before the step of freezing the cells.

In accordance with an exemplary embodiment, one embodiment of the method according to the present disclosure includes the steps, which are entirely performed in vitro. Another embodiment of the method according to the present disclosure includes the steps, which are performed in vivo. Such steps include the collection of cells (such as epidermal cells and blood cells in the case where iPS is used) from the object or the collection of tissues (such as epidermal cells and blood cells in the case where iPS is used) from which cells are supplied. In one embodiment of the method according to the present disclosure, all the steps are performed under aseptic conditions. One embodiment of the method according to the present disclosure is accomplished in such a way that the sheet-shaped cell culture which is eventually obtained is substantially aseptic. One embodiment of the method according to the present disclosure is accomplished in such a way that the sheet-shaped cell culture, which is eventually obtained is aseptic.

Another aspect of the present disclosure relates to a composition, graft, and medical product, each containing the sheet-shaped cell culture defined in the present disclosure. (They will be generally called “Composition etc.” on some occasions.)

The composition etc. defined in the present disclosure may contain, in addition to the sheet-shaped cell culture covered in the present disclosure, a variety of effective components such as pharmaceutically acceptable support, any component that improves the sheet-shaped cell culture in its ability to survive, its ability to stick alive, and/or its various functions, and any component useful for the treatment of the object disease. Such additional components may be known to those skilled in the art. The composition etc. according to the present disclosure may be used in combination with any component that improves the sheet-shaped cell culture in its ability to survive, its ability to stick alive, and/or its various functions, and any other components useful for the treatment of the object disease.

One embodiment is so intended as to use the sheet-shaped cell culture and composition etc. defined in the present disclosure for the treatment of diseases (such as cardiac disease.) In addition, the sheet-shaped cell culture of the present disclosure will be used to produce the composition etc. for the treatment of diseases (such as cardiac disease.) Non-limiting examples of such diseases include cardiac infarction (including chronic cardiac failure), dilated cardiomyopathy, ischemic cardiomyopathy, and cardiac diseases (such as cardiac failure, especially chronic cardiac failure) accompanied by systolic functional disorder (such as left systolic functional disorder.) The diseases may include those to which the cardiocytes and/or sheet-shaped cell culture (cell sheet) are effectively applied.

Another aspect of the present disclosure covers a kit composed of a frozen cell population containing cardiocytes derived from pluripotent stem cells or mesenchymal stem cells (which is obtained by the foregoing method), a cell culture medium, and a culture substrate. (This kit may occasionally be referred as “Kit of the present disclosure” hereinafter.) The culture medium and culture substrate may be selected from the same ones used for culture mentioned above.

In accordance with an exemplary embodiment, the kit of the present disclosure may additionally contain a medical adhesive and a cell washing solution. The medical adhesive is not specifically limited so long as it is one to be used for surgery. Examples of such medical adhesives include those of cyanoacrylate, gelatinaldehyde, and fibrin glue. Preferable among them is fibrin glue adhesive, such as Beriplast® (from CSL Behring K.K.) and Bolheal® (from Teijin Pharma Limited.) The cell washing solution is one which is used in the step of washing the abovementioned cells.

The kit of the present disclosure may additionally contain one or two or more cells (selected from vascular endothelial cells, mural cells, and fibroblasts), the foregoing additives, culture dish, reagents (such as antibody, washing solution, and beads) to be used for purification of cardiocytes, tools (such as pipette, dropper, and tweezers), and instructions for the method of producing and using the sheet-shaped cell culture. Such instructions may be available in the form of any medium recording them, such as flexible disc, CD, DVD, Blu-ray disc, memory card, and USB memory.

Another aspect of the present disclosure is intended for drug screening with the help of the sheet-shaped cell culture of the present disclosure, the composition of the present disclosure, the kit of the present disclosure, and the sheet-shaped cell culture of the present disclosure. The sheet-shaped cell culture of the present disclosure may be used in place of laboratory animals, which were conventionally used for drug screening. The kind of drug to be screened and the method for screening may be properly selected and established by those who are skilled in the art.

Another aspect of the present disclosure is directed to the method of treating the disease of the object by application to the object with an effective amount of the sheet-shaped cell culture or composition of the present disclosure. The diseases for treatment have been mentioned above in relation to the sheet-shaped cell culture or composition of the present disclosure.

The term “object” used in the present disclosure denotes any living organism, preferably animal, more preferably mammal, and most desirably human individuals. The object used in the present disclosure may be healthy ones or sick ones. In the case where the treatment of diseases relating to the anomaly of tissue is intended, the object means the one which suffering from the disease or the one which is liable to suffer from the disease.

The term “treatment” embraces any intervention for prevention and/or therapy which are medically allowed for therapy, prevention, and temporary abatement. In other words, the term “treatment” embraces medically allowable interventions for various purposes, which are intended to retard or suspend the progress of disease relating to the anomaly of tissues, the regression or disappearance of lesion, and the prevention of disease or the prevention of recurrence of disease.

In accordance with an exemplary embodiment, the method for treatment according to the present disclosure may be accomplished by using the sheet-shaped cell culture or composition of the present disclosure in combination with any component that allows the sheet-shaped cell culture to improve in its ability to survive, its ability to stick alive, and/or its various functions, or with any effective component useful for treatment of the object disease.

The method for treatment according to the present disclosure may further include the step of producing the sheet-shaped cell culture of the present disclosure by means of the method for production according to the present disclosure. The method for treatment according to the present disclosure may include an additional step to be performed before the step of producing the sheet-shaped cell culture. The additional step is designed to collect cells (such as epidermal cells and blood cells in the case where iPS is used) from the object for the production of the sheet-shaped cell culture or to collect tissues (such as epidermal cells and blood cells in the case where iPS is used) from which cells are supplied. In one embodiment, the object from which cells are collected or tissues as a source of cells are collected is identical with the one, which receives the sheet-shaped cell culture or composition etc. According to another embodiment, the object from which cells are collected or tissues as a source of cells are collected is a different individual of the species, which receives the sheet-shaped cell culture or composition etc. According to further another embodiment, the object from which cells are collected or tissues as a source of cells are collected is an individual of different species, which receives the sheet-shaped cell culture or composition etc.

In accordance with an exemplary embodiment, the term “effective amount” used in the present disclosure denotes an amount (for example, the size, weight, number) enough to suppress the onset and recurrence of diseases, to alleviate symptom, or to retard or suspend the progress of symptom. It should preferably be an amount enough to suppress the onset and recurrence of diseases or enough to cure the diseases. In addition, the effective amount should be small enough not to cause an adverse effect that offsets the advantage resulting from administration. Such an effective amount may be appropriately established by experiments with laboratory animals (such as mouse, rat, dog, and pig) or disease model animals. Methods for such experiments are known to those skilled in the art. The effective amount may be established based mainly on the size of the tissue lesion to which treatment is applied.

A typical method for administration is by the direct application to the tissue. The frequency of administration is once typically for one treatment, but administration may be repeated until the desired effect is attained. Application to the tissue may be facilitated with the help of fastening means (such as thread and staple) which fixes the sheet-shaped cell culture or composition of the present disclosure to the tissue.

In accordance with an exemplary embodiment, another aspect of the present disclosure includes the method of improving the purity of differentiated cardiocytes which have been derived from pluripotent stem cells or mesenchymal stem cells derived from adipose tissue or bone marrow, the method including a step of freezing cells which have dissociated from a cell population which has undergone differentiation and induction to cardiocytes from pluripotent stem cells or mesenchymal stem cells derived from adipose tissue or bone marrow in a cryopreserving solution containing a cryoprotective agent.

The step of freezing just mentioned above may be performed in the same way as described in the method of production according to the present disclosure. The term “improving the purity of differentiated cardiocytes which have been derived from pluripotent stem cells or mesenchymal stem cells derived from adipose tissue or bone marrow” means that the ratio of the number of cardiocytes derived from differentiated pluripotent stem cells or mesenchymal stem cells to the number of all the cells which have dissociated from the cell population which has been induced to differentiation into cardiocytes from pluripotent stem cells or mesenchymal stem cells increases after freezing by, for example, equal to or more than approximately 5%, equal to or more than approximately 10%, equal to or more than approximately 15%, or equal to or more than approximately 20%.

In accordance with an exemplary embodiment, another aspect of the present disclosure includes the method of reducing the ratio of undifferentiated pluripotent stem cells or mesenchymal stem cells derived from adipose tissue or bone marrow in the dissociated cells mentioned above, the method including a step of freezing cells which have dissociated from a cell population which has undergone differentiation and induction to cardiocytes from pluripotent stem cells or mesenchymal stem cells derived from adipose tissue or bone marrow in a cryopreserving solution containing a cryoprotectant.

The step of freezing just mentioned above may be performed in the same way as described in the method of production according to the present disclosure. The term “reducing the ratio of undifferentiated cardiocytes which have been derived from pluripotent stem cells or mesenchymal stem cells derived from adipose tissue or bone marrow” means that the ratio of the number of cardiocytes derived from differentiated pluripotent stem cells or mesenchymal stem cells to the number of all the cells which have dissociated from the cell population which has been induced to differentiation into cardiocytes from pluripotent stem cells or mesenchymal stem cells decreases after freezing by, for example, equal to or more than approximately 5%, equal to or more than approximately 10%, equal to or more than approximately 15%, or equal to or more than approximately 20%.

### EXAMPLES

The present disclosure will be described below in more detail with reference to the specific examples which are not intended to restrict the scope thereof.

### Example 1—Induction of Human iPS Cells into Cardiocytes

This example relies on human iPS cells (strain 253G1) purchased from Riken (Institute of Physical and Chemical Research.) The human iPS cells underwent differentiation and induction into cardiocytes by means of a reactor according to the method described in Matsuura K et al., Biochem Biophy Res Commun, 2012 Aug. 24; 425(2): 3217. To be more specific, the procedure includes the following steps. The 253G1 cells (undifferentiated) were cultured on MEF treated with mitomycin C, with Primate ES culture medium (from ReproCELL Inc.) incorporated with bFGF (5 ng/mL) being used as the culture medium to maintain undifferentiation. The undifferentiated 253G1 cells were recovered with the help of peeling solution (from ReproCELL Inc.) from ten culture dishes (10 cm.) The recovered cells were suspended in 100 mL of mTeSR culture medium (from STEMCELL Technologies Inc.) incorporated with 10 μM of Y27632 (ROCK inhibitor.) The resulting suspension was transferred to a vessel, which was subsequently placed in a bioreactor (from Able Corporation) for spinner culture. After 1 day, the culture medium was freed of Y27632. After 1 to 3 days, the culture medium was replaced by StemPro34 (from Life Technologies Corporation.) After 3 to 4 days, BMP4 (0.5 ng/mL) was added. After 4 to 7 days, there were added BMP4 (10 ng/mL), bFGF (5 ng/mL), and activin A (3 ng/mL.) After 7 to 9 days, IWR1 (4 μM) was added. After 9 days, VEGF (5 ng/mL) and bFGF (10 mg/mL) were added, and stirring was continued. After 16 to 18 days, the cells were recovered.

In this way, there was obtained a cell population (cell mass) containing cardiocytes derived from human iPS cells. The thus obtained cell population was dissociated by trypsin/EDTA (0.05%) and purified of remaining cell aggregates by a strainer (from BD Biosciences) to be used for the ensuing experiments.

### Example 2—Cryopreservation and Thawing of Cardiocytes Derived from Human iPS Cells

A portion of the dissociated cell population obtained in Example 1 was suspended in a serum replacement for cell culture containing 10% DMSO to give a suspension of concentration ranging from 2.5×10⁶ cells/mL to 1.1×10⁷ cells/mL. The suspension underwent cryopreservation in a liquid nitrogen tank.

The frozen cells were thawed at 37° C. and then washed twice with a buffer solution containing serum albumin (0.5%).

### Example 3—Evaluation of Survival Ratio of Cardiocytes Derived from Human iPS Cells (Part 1)

There were prepared four samples of cells which underwent the freeze-thaw process individually as mentioned in Example 2.

A portion of cells was collected from each sample, and live cells and dead cells were counted by trypan blue staining. The number of live cells and dead cells was used to determine the survival ratio. The results are depicted in Table 1 below.

TABLE 1
Cell Survival Ratio

| Sample | Survival Ratio |
|---|---|
| 1 | 95% |
| 2 | 99% |
| 3 | 91% |
| 4 | 98% |

It is noted from Table 1 that the cardiocytes derived from pluripotent stem cells maintained a high survival ratio after the freeze-thaw process which was carried out according to the method of the present disclosure.

### Example 4—Evaluation of Survival Ratio of Cardiocytes Derived from Human iPS Cells (Part 2)

The cells obtained in Example 1 were examined for the ratio between iPS cells and cardiocytes contained therein in the following manner. First, a portion of the cells was double-labeled with SSEA4 or Tra160 (marker for iPS cells) and cTNT (marker for cardiocytes.)

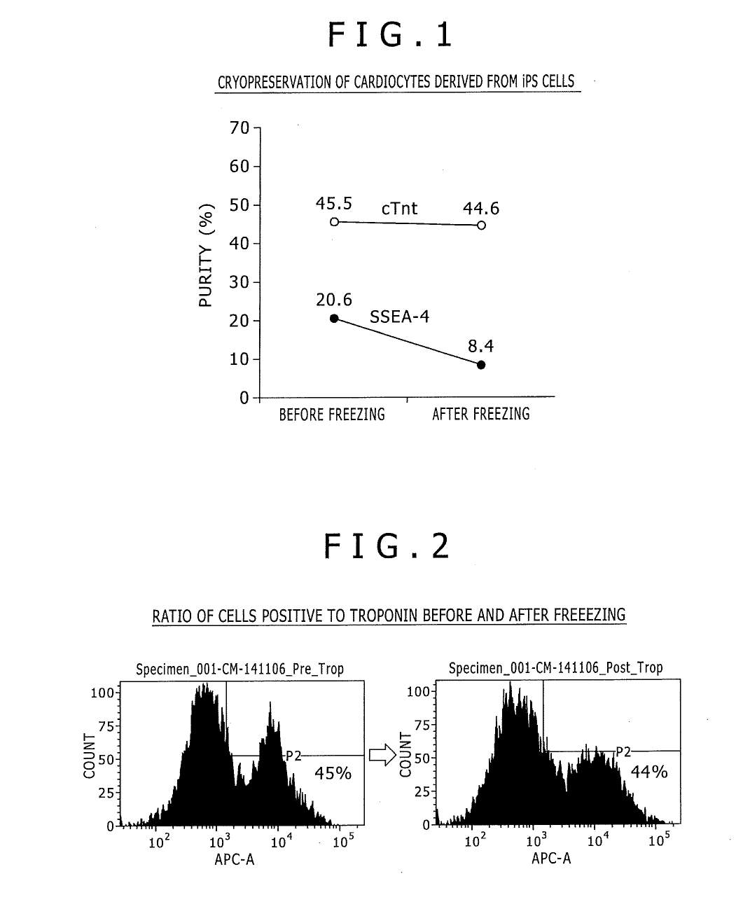

The labeled cells were analyzed by means of the BD FACSCanto™ II flowcytometer and the BD FACSDiva™ software (either of the two made by BD Bioscience Inc.) The ratio of the number of iPS cells to the number of entire cells, and the ratio of the number of cardiocytes to the number of entire cells were counted respectively in terms of the ratio of the number of cells positive to SSEA-A or Tra160 to the number of entire cells and the ratio of the number of cells positive to cTNT to the number of entire cells. The results are depicted in FIGS. 1 to 3.

For the purpose of reference, a portion of the iPS cells (not yet induced to differentiation) used in Example 1 was dissociated and freed of cell aggregates by the method mentioned in Example 1 and then underwent freezing and thawing by the method mentioned in Example 2. The thus obtained cells underwent the same procedure as in Example 2 for labeling, analysis, and calculation. The results are depicted in FIG. 4.

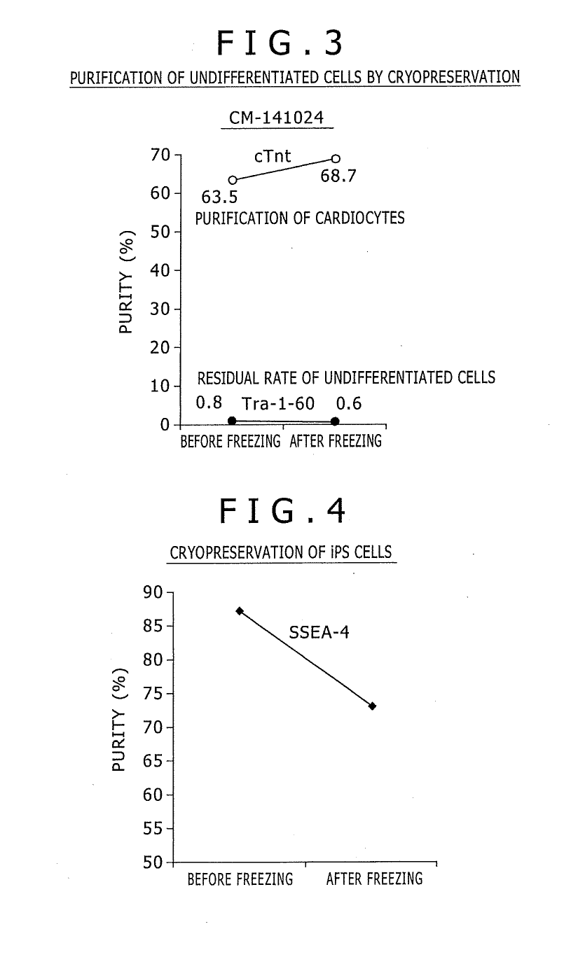

It is noted from FIGS. 1 and 2 that, in the case of cells double-labeled with SSEA4 and cTNT, the ratio of cells positive to SSEA4 decreased from 20.6% (before freezing) to 8.4% (after freezing), whereas the ratio of cells positive to cTNT only slightly decreased from 45% level (before freezing) to 44% level (after freezing.) It is also noted from FIG. 4 that the ratio of cells positive to SSEA4 decreased from approximately 87% to approximately 73%. It is also noted from FIG. 3 that the ratio of cells positive to Tra160 decreased from 0.8% (before freezing) to 0.6% (after freezing) (with the ratio of decrease being 25%), whereas the ratio of cells positive to cTNT increased from 63.5% (before freezing) to 68.7% (after freezing.)

### Example 5—Preparation of Sheet-Shaped Cell Culture

A culture medium containing 20% serum was added to a 3.5 cm dish (UpCell® from CellSeed Inc.) to such an extent that it covers the surface for culture almost entirely. The dish was kept covered with the culture medium for 3 hours to 3 days at 37° C. in an environment of 5% CO₂. After this step, the culture medium added to the dish was discarded. The thus treated dish (UpCell®) was inoculated with the cells obtained in Example 2, which had been suspended in a culture medium containing 10% serum. The density of cells inoculated was 2×10⁵/cm² to 10×10⁵/cm². This step was followed by cultivation for 3 to 5 days at 37° C. in an environment of 5% CO₂ to make a sheet-shaped cell culture.

### Example 6—Evaluation of the Sheet-Shaped Cell Culture

### (1) Appearance

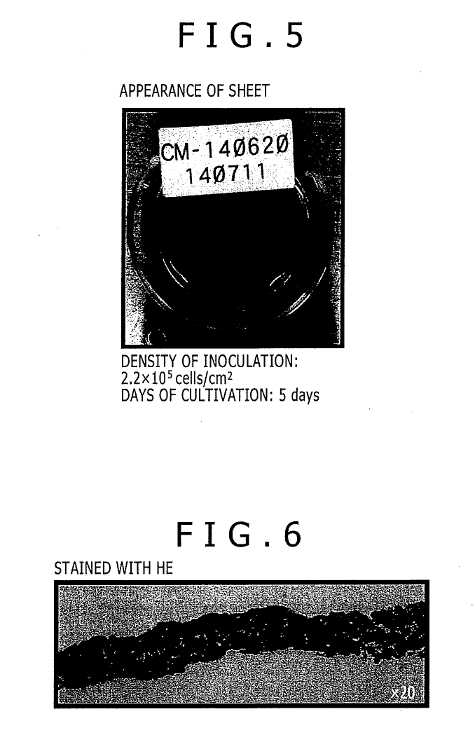

It was found that the sheet-shaped cell culture obtained in Example 5 took on a white circular form suitable for transplantation as depicted in FIG. 5.

(2) Staining with Hematoxylin Eosin

A portion of the cells of the sheet-shaped cell culture obtained in Example 5 was collected, and the collected cells were stained with hematoxylin eosin and the stained cells were observed under an optical microscope. It is noted from FIG. 6 that cell nuclei were stained bluish violet and cytoplasm was stained reddish yellow; this indicates that the cells constituting the sheet-shaped cell culture obtained in Example 5 have the normal cell membranes and cell nuclei.

### (3) Immunostaining

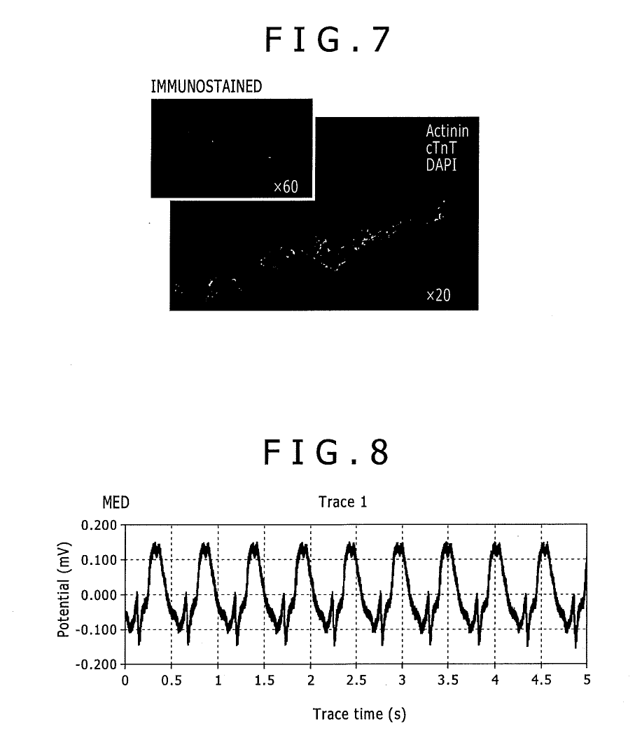

A portion of the cells of the sheet-shaped cell culture obtained in Example 5 was collected, and the collected cells underwent multiple staining with Actinin (marker for actinin), c-TNT (marker for cardiocytes) and 4′,6diamidino2phenylindole (DAPI) (marker for DNA.) The thus labeled cells were excited for fluorescence emission and observed under a fluorescent microscope. As depicted in FIG. 7, that part positive to Actinin emitted a red color, that part positive to c-TNT emitted a green color, and that part positive to DAPI emitted a bluish violet color. FIG. 7 indicates that the cells constituting the sheet-shaped cell culture obtained in Example 5 are cardiocytes and function as cardiocytes.

### (4) Synchronous Pulsation

The sheet-shaped cell culture obtained in Example 5 was examined by means of the multichannel extracellular recording method (Multi Electrode Dish: MED, MED 64 system from Alpha Med Scientific Inc.) The sheet-shaped cell culture gave spontaneous synchronous pulsation, as depicted in FIG. 8.

### Example 7—Data Suggesting Stability of Cardiocytes Derived from Human iPS Cells

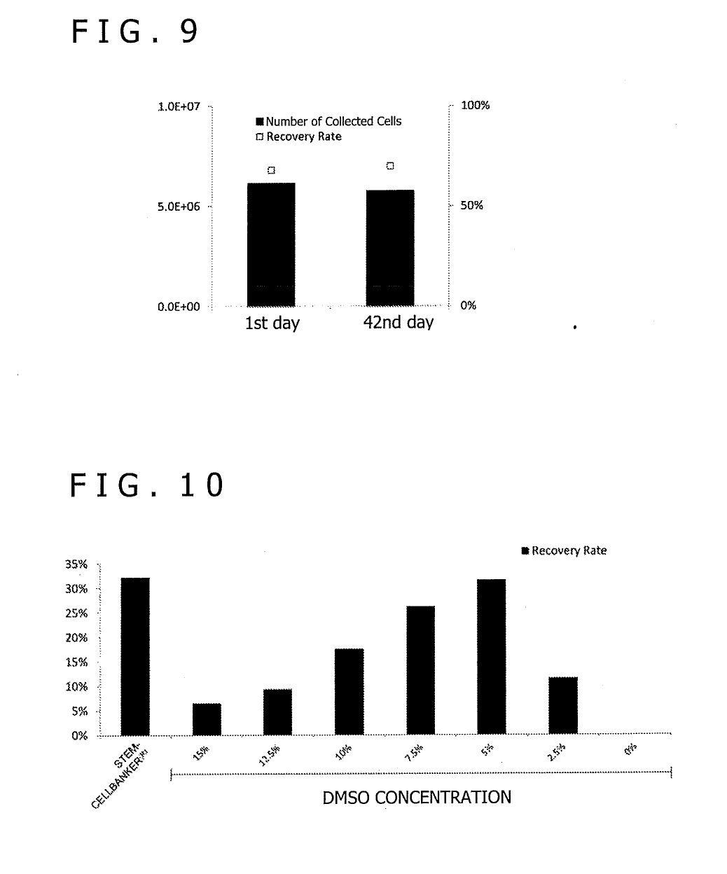

A portion of the dissociated cell population obtained in Example 1 was suspended in STEM-CELLBANKER® GMP Grade (commercially available from Nippon Zenyaku Kogyo Co., Ltd.) The density of suspension was 1.0×10⁷/mL. The resulting suspension was slowly frozen by means of a program freezer PDF2000G (from Strex Inc.) and cryopreserved in a liquid nitrogen tank. The slow freezing by the program freezer started with preconditioning at 4° C. for 10 minutes and then proceeded at a cooling rate of −1° C./minute. The frozen cells were thawed at 37° C. on the first and 42nd days counting from the day on which the cryopreservation started. The thawed cells were washed twice with a buffer solution containing 0.5% serum albumin. Cells were collected from each sample, and the collected cells underwent trypan blue staining to count live cells and dead cells. The cell recovery ratio was calculated from the total number of cells and the number of live cells. The results are depicted in FIG. 9. It is noted from FIG. 9 that the cardiocytes derived from pluripotent stem cells which have underwent cryopreservation and thawing according to the method of the present disclosure maintain the cell recovery ratio of approximately 70% regardless of whether thawing was performed on the first day or 42nd days after the start of cryopreservation. The foregoing result indicates that the cardiocytes derived from pluripotent stem cells which have underwent cryopreservation and thawing according to the method of the present disclosure remain highly stable even after storage for equal to or more than one month.

### Example 8—Comparison of Cryopreserving Solutions

A portion of the dissociated cell population obtained in Example 1 was suspended in a culture medium containing 0% to 15% DMSO in serum replacement or in STEM-CELLBANKER® GMP Grade (commercially available from Nippon Zenyaku Kogyo Co., Ltd.) The density of suspension was 1.0×10⁶/mL. The suspension underwent slow freezing with BICELL® at −80° C. in an ultra-deep freezer and then stored in a liquid nitrogen tank. Subsequently, the cryopreserved cells were thawed at 37° C. and washed twice with a buffer solution containing 0.5% serum albumin. Cells of each sample were collected and underwent trypan blue staining to count live cells and dead cells. The cell recovery ratio was calculated from the total number of cells and the number of live cells. The results are depicted in FIG. 10, which indicates that the cryopreserving solution commercially available as STEM-CELLBANKER® GMP Grade and the cryopreserving solution containing DMSO (5% to 10%) gave the recovery ratio of cardiocytes equal to or higher than 25%.

### Example 9—Comparison of Freezing Methods

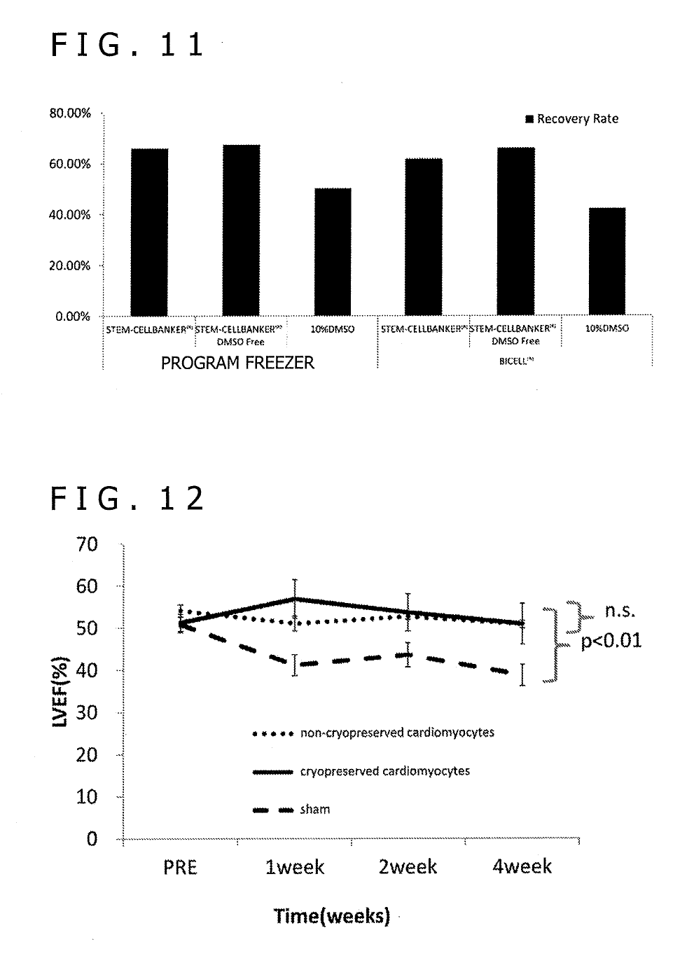

A portion of the dissociated cell population obtained in Example 1 was suspended in a culture medium containing 10% DMSO in serum replacement or in STEM-CELLBANKER® GMP Grade or STEM-CELLBANKER® DMSO Free GMP Grade (commercially available from Nippon Zenyaku Kogyo Co., Ltd.) The density of suspension was 1.0×10⁶/mL. The suspension underwent slow freezing with program freezer PDF2000G (Strex Inc.) or with BICELL® at −80° C. in an ultra-deep freezer and then stored in a liquid nitrogen tank. The slow freezing by the program freezer started with preconditioning at 4° C. for 10 minutes and then proceeded at a rate of −1° C./minute. Subsequently, the cryopreserved cells prepared with the help of variously combined cryopreserving solutions and freezing means were thawed at 37° C. and washed twice with a buffer solution containing 0.5% serum albumin. Cells of each sample were collected and underwent trypan blue staining to count live cells and dead cells. The cell recovery ratio was calculated from the total number of cells and the number of live cells. The results are depicted in FIG. 11, which indicates that the recovery ratio of cardiocytes was equal to or higher than 42% regardless of combinations. The particularly high recovery ratio (67%) was achieved in the case where the cryopreservation was carried out with the help of STEM-CELLBANKER® DMSO Free GMP Grade and program freezer.

### Example 10—Evaluation of Effectiveness of Sheet of Cardiocytes Derived from Human iPS Cells

Cardiocytes derived from iPS cells were made into a cell sheet after freezing or without freezing. Each sample of the resulting cell sheets was transplanted to a nude rat suffering from ischemic cardiac infarction to see if it is effective in improving the cardiac function. The freezing of the cardiocytes derived from iPS cells was accomplished in the following manner. A portion of the cardiocytes derived from iPS cells to be frozen was suspended in STEM-CELLBANKER® GMP Grade (from Nippon Zenyaku Kogyo Co., Ltd.) The density of suspension was 1.0×10⁷ cells/mL. The resulting suspension was slowly frozen by means of a program freezer and cryopreserved in a liquid nitrogen tank. The frozen cells were thawed at 37° C. and then washed twice with a buffer solution containing 0.5% serum albumin. A portion of the cardiocytes derived from iPS cells not to be frozen was suspended in a culture medium containing 20% serum. The suspended cells were collected and underwent trypan blue staining to calculate the ratio of cell recovery from the total number of cells and the number of live cells. A culture medium containing 20% serum was added to an UpCell® 48well dish (from CellSeed Inc.) to such an extent that it covers the surface for culture almost entirely. The dish was kept covered with the culture medium for 3 hours to 3 days at 37° C. in an environment of 5% CO₂. After this step, the culture medium added to the dish was discarded. Each sample of the frozen cardiocytes derived from iPS cells and the unfrozen cardiocytes derived from iPS cells was suspended in a culture medium containing 10% serum. Each of the resulting suspensions was inoculated onto the treated UpCell® with a density of 2×10⁵ cells/cm² to 10×10⁵ cells/cm². Cultivation was performed for 2 to 5 days at 37° C. in an environment of 5% CO₂, so that the resulting culture took on a sheet-like form. The resulting sheet of cells was peeled off from the dish and then transplanted to the surface of the heart of a nude rat suffering from ischemic cardiac infarction. The results are depicted in FIG. 12. It was found from echocardiography and cardiac catheterization on fourth week after transplantation that the group of rats which received transplantation with a sheet of cardiocytes derived from iPS cells significantly improved in cardiac function (in terms of ejection fraction: EF, and fractional shortening: FS) as compared with the Sham operation group which did not receive transplantation of a sheet-shaped cell culture (Group with non-frozen cardiocytes: 51±3%, n=6, Group with frozen cardiocytes: 51±5%, n=7, Sham group: 39±1%, n=6.) On the other hand, no significant difference was noticed in cardiac function between the group with non-frozen cardiocytes and the group with frozen cardiocytes. Neither of the two groups suffered from particular anomaly, such as tumorigenesis, due to the transplanted cell sheet was found.

The specification given herein discloses a variety of features of the present disclosure, which can be variously combined together to constitute varied embodiments which are not specifically described in this specification but are embraced in the scope of the present disclosure. It is known to those skilled in the art that such embodiments can be variously changed and modified within the spirit and scope of the present disclosure and that any equivalent of such modifications is also included in the scope of the present disclosure. Consequently, it is to be understood that the embodiments disclosed herein are merely examples and they are not intended to restrict the scope of the present disclosure.

The detailed description above describes a method for cryopreservation of cardiocytes derived from pluripotent stem cells or mesenchymal stem cells derived from adipose tissue or bone marrow, a method for producing a sheet-shaped cell culture containing the cardiocytes, and an application of the sheet-shaped cell culture. The invention is not limited, however, to the precise embodiments and variations described. Various changes, modifications, and equivalents can be effected by one skilled in the art without departing from the spirit and scope of the invention as defined in the accompanying claims. It is expressly intended that all such changes, modifications, and equivalents which fall within the scope of the claims are embraced by the claims.

What is claimed is:

1. A method for cryopreservation of cardiocytes derived from pluripotent stem cells or mesenchymal stem cells derived from adipose tissue or bone marrow, the method comprising:
dissociating a cell population which has been induced to differentiate into cardiocytes from pluripotent stem cells or mesenchymal stem cells derived from adipose tissue or bone marrow to cells.

2. The method for cryopreservation according to claim 1, further comprising:
freezing the dissociated cells in a cryopreservation solution which contains a cryoprotectant.

3. The method for cryopreservation according to claim 2, wherein the cryoprotectant is one capable of permeation through cell membrane.

4. The method for cryopreservation according to claim 2, wherein the cryoprotectant is one or more species selected from the group consisting of dimethyl sulfoxide, ethylene glycol, propylene glycol, 1,2-propanediol, 1,3-propanediol, butylene glycol, isopropylene glycol, dipropylene glycol, and glycerin.

5. The method for cryopreservation according to claim 4, wherein the cryoprotectant is dimethyl sulfoxide.

6. The method for cryopreservation according to claim 4, wherein the cryoprotectant is 1,2-propanediol.

7. The method for cryopreservation according to claim 1, wherein the pluripotent stem cells are induced pluripotent stem cells.

8. A method for producing a sheet-shaped cell culture comprising:
thawing the frozen cells which are obtained by the method defined in claim 2; and
forming the thawed cells into a sheet-shaped cell culture.

9. The method for drug screening, comprising:
using the sheet-shaped cell culture produced by the method according to claim 8 or a composition comprising the sheet-shaped cell culture.

10. The method for drug screening, comprising:
using a kit comprising the frozen cells obtained by the method according to claim 1, a cell culture solution, and a culture substrate.

11. The method for cryopreservation to claim 10, wherein the kit further comprises:
a medical adhesive and a cell washing solution.

12. A method for treating a disease in a patient comprising:
applying an effective amount of the sheet-shaped cell culture produced by the method according to claim 8 or a composition containing the sheet-shaped cell culture to the patient in need thereof.

13. A method for increasing the purity of differentiated cardiocytes derived from pluripotent stem cells or mesenchymal stem cells derived from adipose tissue or bone marrow in the cells dissociated from the cell population which has been induced to differentiate into cardiocytes from pluripotent stem cells or mesenchymal stem cells derived from adipose tissue or bone marrow, the method comprising:
freezing the dissociated cells in a cryopreserving solution containing a cryoprotectant.

14. The method according to claim 13, wherein the cryoprotectant is one capable of permeation through cell membrane.

15. The method according to claim 13, wherein the cryoprotectant is one or more species selected from the group consisting of dimethyl sulfoxide, ethylene glycol, propylene glycol, 1,2-propanediol, 1,3-propanediol, butylene glycol, isopropylene glycol, dipropylene glycol, and glycerin.

16. The method according to claim 15, wherein the cryoprotectant is dimethyl sulfoxide or 1,2-propanediol.

17. A method for decreasing the ratio of undifferentiated pluripotent stem cells or mesenchymal stem cells derived from adipose tissue or bone marrow in the cells dissociated from the cell population which has been induced to differentiate into cardiocytes from pluripotent stem cells or mesenchymal stem cells derived from adipose tissue or bone marrow, the method comprising:
freezing the dissociated cells in a cryopreserving solution containing a cryoprotectant.

18. The method according to claim 17, wherein the cryoprotectant is one capable of permeation through cell membrane.

19. The method according to claim 17, wherein the cryoprotectant is one or more species selected from the group consisting of dimethyl sulfoxide, ethylene glycol, propylene glycol, 1,2-propanediol, 1,3-propanediol, butylene glycol, isopropylene glycol, dipropylene glycol, and glycerin.

20. The method according to claim 19, wherein the cryoprotectant is dimethyl sulfoxide or 1,2-propanediol.
